(19) United States
(12) Patent Application Publication
(10) Pub. No.: US 2013/0296441 A1
(43) Pub. Date: Nov. 7, 2013

(54) Anti-Obesity Properties of Pterostilbene

(71) Applicants: Agnes Rimando, Oxford, MS (US); Maria del Puy Portillo, Vitoria (ES)

(72) Inventors: Agnes Rimando, Oxford, MS (US); Maria del Puy Portillo, Vitoria (ES)

(21) Appl. No.: 13/463,442

(22) Filed: May 3, 2012

(51) Int. Cl. A61K 31/09; A61P 3/04

(52) U.S. Cl. 514/720

(57) ABSTRACT

We have determined that pterostilbene demonstrates anti-obesity properties. Rats were fed a commercial obesogenic diet supplemented or not with pterostilbene: PT15 (low dose: 15 mg/kg body weight/d) or PT30 (high dose: 30 mg/kg body weight/d) for 6 weeks. Pterostilbene significantly reduced total adipose tissue mass (15.1% in the PT15 group; 22.9% in the PT30 group) without changes in food intake. The activities of lipogenic enzymes were reduced in white adipose tissue and liver. Activities of enzymes involved in liver fatty acid oxidation were significantly increased in the PT30 group. Low dose pterostilbene can reduce body fat by reducing lipogenesis in adipose tissue. At the high dose, two mechanisms contribute to reduction in body fat: (1) decrease in adipose tissue and liver lipogenesis, and (2) an increase in liver fatty acid oxidation.

### BACKGROUND OF THE INVENTION

1. Field of the Invention

This invention relates to a method for treating, alleviating or preventing obesity by administering a pharmaceutical composition comprising a therapeutically effective amount of pterostilbene.

2. Description of the Relevant Art

Obesity and overweight are major public health concerns spreading throughout the world across all age sectors, afflicting not only adults but also children and adolescents. Moreover, obesity is associated with several chronic diseases, such as diabetes, stroke, and hypertension (James et al. 2004. Eur. J. Cardiovasc. Prev. Rehabil. 11:3-8). Considerable efforts are being made to identify and characterize novel naturally-occurring molecules which are orally active and safe and can be employed for obesity management, using a broad range of in vivo and in vitro methodologies. Phenolic compounds make up one of the groups of molecules that have been most frequently studied in recent years.

Pterostilbene is a phenolic compound biologically classified as a phytoalexin, which is an antimicrobial substance that is part of a plant's defense system and is synthesized in response to pathogen infection, as well as to excessive ultraviolet exposure (Bavaresco et al. 1999. Drugs Exp. Clin. Res. 25:57-63). Pterostilbene is known to have diverse benefits for the prevention and treatment of wide variety of diseases, including cancer (Rimando et al. 2008. Planta Med. 74:1635-1643; Pan et al. 2009. Carcinogenesis 30:1234-1242; Paul et al. 2010. Carcinogenesis 31:1272-1278), dyslipidemia (Rimando et al. 2005. J. Agric. Food Chem. 53:3403-3407; Sateesh and Pari. 2008. J. Appl. Biomed. 6:31-37), diabetes (Sateesh and Pari. 2006. J. Pharm. Pharmacol. 58:1483-1490), cardiovascular disease (Park et al. 2010. Vascular Pharmacol. 53:61-67) and cognitive function degeneration (Joseph et al. 2008. J. Agric. Food Chem. 56:10544-10551).

Pterostilbene is not known to be toxic or cause adverse effects in humans. In mice fed this phenol for 28 days at doses up to 3000 mg/kg body weight/d, equivalent to 500 times the estimated mean human intake, no significant toxic or adverse biochemical effects were noted, compared to controls (Ruiz et al. 2009. J. Agric. Food Chem. 57:2130-3186).

There is a need for agents that can be used to treat obesity. The aim of the present research was to determine whether pterostilbene demonstrates anti-obesity properties.

### SUMMARY OF THE INVENTION

We have investigated the property of pterostilbene as an inhibitor of obesity and have determined that pterostilbene can be used as an anti-obesity agent.

In accordance with this discovery, it is an object of the invention to provide method for treating, alleviating or preventing obesity in a subject in need thereof by administering a therapeutically effective dose of pterostilbene, its pharmaceutically acceptable salts or isomers thereof.

It is an additional object of the invention to provide a method of decreasing the total adipose tissue mass in an individual.

It is another object of the invention to provide a method of reducing depots of adipose tissue in particular anatomical locations, namely, subcutaneous and/or mesenteric and/or perirenal depots.

It is a further object of the invention to provide a method of inhibiting lipogenesis in an individual.

It is still another object of the invention to provide a method of increasing fatty acid oxidation in the liver of an individual.

Also part of this invention is a kit, comprising a pharmaceutical composition containing pterostilbene; and instructions for the use of the kit.

Other objects and advantages of this invention will become readily apparent from the ensuing description.

### BRIEF DESCRIPTION OF THE DRAWINGS

FIG. 1 shows the chemical structure of pterostilbene.

FIG. 2 shows the general scheme for lipid synthesis showing enzymes affected by pterostilbene (retrieved and adapted from the Internet: <URL: www.jbc.org/content/281/39/28721/F2.large.jpg).

### DETAILED DESCRIPTION OF THE INVENTION

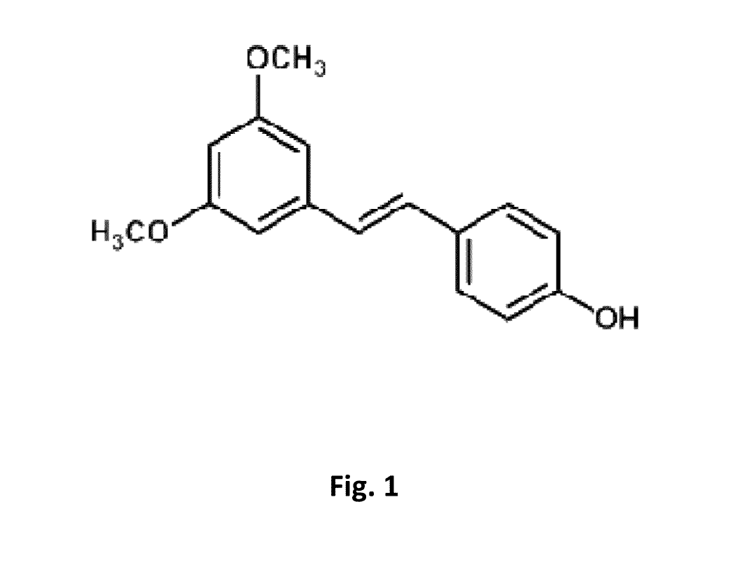

We evaluated the anti-obesity properties of pterostilbene (see FIG. 1) by measuring its effects on body fat in rats fed hypercaloric diets, by assessing the mechanisms of action responsible for anti-obesity effects and by analyzing levels of pterostilbene in serum and tissues. Results showed that pterostilbene prevented accumulation of body fat induced by high-fat and high-sucrose feeding. Two doses of pterostilbene (15 and 30 mg/kg BW/d) were used in order to determine whether there would be a dose-response effect. The anti-obesity effect of the higher dose was greater than that of the lower dose, i.e., there was a 22.9% decrease in total adipose tissue mass in the group given the higher dose (30 mg/kg BW/d) and a 15.1% decrease in the lower dose group (15 mg/kg BW/d); however, significant differences between these two groups were not found. Consistent with this observed effect, the higher dose group also showed higher levels of pterostilbene in the serum, liver, epididymis and subcutaneous tissues.

As far as specific anatomical locations are concerned, the PT15 dose mainly affected subcutaneous depots; whereas, the PT30 dose reduced both subcutaneous and internal (mesenteric and perirenal) depots. It is important to note that internal depots are more closely related to insulin resistance. It is also interesting that pterostilbene was found in muscle of the PT30 group.

The reduction in fat accumulation induced by pterostilbene was not due to a decrease in food intake (see Table I). Thus, in order to find potential mechanisms underlying the body fat decrease, the effects of pterostilbene treatment on several metabolic pathways were assessed.

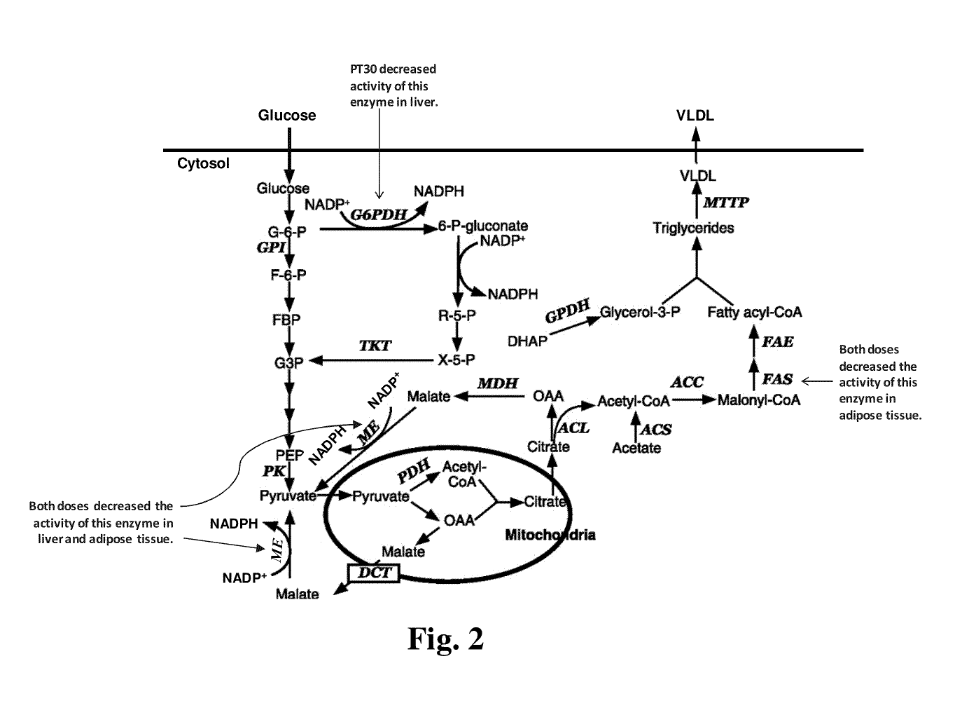

The amount of triacylglycerol stored in adipose tissue results from the balance among (1) de novo lipogenesis, (2) fatty acid uptake from circulating triacylglycerols, and (3) lipid mobilization. Those fatty acids, which are synthesized via de novo lipogenesis from acetyl-CoA and glycerol-3-phosphate coming from glucose metabolism, form triacylglycerols which are stored in adipose tissue to be used during periods of food deprivation (Vernon and Taylor. 1986. J. Anim. Sci. 63:1119-1125; Hillgartner et al. 1995. Physiol. Rev. 75:47-76). Adipose tissue can also obtain fatty acids from circulating triacylglycerols (chylomicrons and very low density lipoprotein [VLDL]). This fatty acid uptake is mediated by heparin-releasable lipoprotein lipase (HR-LPL), the active form of lipoprotein lipase (LPL), which is located in the adipose tissue endothelium (Fielding and Frayn. 1998. Br. J. Nutr. 80:495-502). In the present study one of these two metabolic routes, i.e., lipogenesis, was inhibited by both pterostilbene doses (see FIG. 2). This provides an explanation for the body fat lowering effect of this phenolic compound. Pterostilbene had no effect on the activity of HR-LPL; thus, indicating that it had no effect on fatty acid uptake.

The accumulation of fat in adipose tissue depends not only on lipid metabolism in this particular tissue, but also on lipid metabolism in other tissues and organs, such as the liver. Pterostilbene was detected in the liver, epididymis and subcutaneous tissues. Therefore pterostilbene appears to have a direct effect on lipid metabolism in adipose tissue as well as indirect effect from metabolism occurring in the liver. Lipogenesis and fatty acid oxidation are important metabolic pathways in triacylglycerol metabolism in the liver. Although pterostilbene, at a dose of 30 mg/kg BW/d, significantly reduced the activity of hepatic malic enzyme and glucose-6-phosphate dehydrogenase, enzymes involved in the production of NADPH, the activity of hepatic fatty acid synthase, a more rate-limiting enzyme (Kim, K. H. 1997. Annu. Rev. Nutr. 17:77-99), was not significantly modified (see Table III). Thus, reduced amounts of triacylglycerols that have originated in the liver and are observed in the plasma, and are available for uptake by the adipose tissue, do not contribute to the anti-obesity effects of pterostilbene.

In order to analyze the effects of pterostilbene on fatty acid oxidation in the liver, the activities of carnitine palmitoyltransferase-I (CPT) and acyl-coenzyme A oxidase (ACO), two rate-limiting oxidative enzymes, were measured. The higher dose of pterostilbene significantly increased the activity of both enzymes. The higher dose group showed higher levels of pterostilbene in the serum and liver, which could explain the greater activity observed in the PT30 group compared to the lower dose group. It is well known that an increase in the activity of these enzymes is a mechanism of action underlying the anti-obesity action of several molecules in animal models, because under these metabolic conditions fatty acids are channeled towards oxidative routes instead of towards storage routes (Macarulla et al. 2005. Nutrition 21:512-519).

Thus, with the lower dose, pterostilbene can reduce body fat by reducing lipogenesis in the adipose tissue. With the higher dose of pterostilbene, the two mechanisms of action that contribute to the reduction in body fat accumulation are: (1) the reduction in adipose tissue lipogenesis and (2) an increase in activities of hepatic CPT and ACO, thus increasing fatty acid oxidation. These mechanisms explain the anti-obesity effect and the greater decrease in adipose tissue size resulting from the higher, compared to the lower, dose of pterostilbene.

A composition in accordance with the present invention containing pterostilbene, or a pharmaceutically acceptable salt of pterostilbene, can be prepared by conventional procedures for blending and mixing compounds. Preferably, the composition also includes an excipient, most preferably a pharmaceutical excipient. Compositions containing an excipient and incorporating the pterostilbene can be prepared by procedures known in the art. For example, pterostilbene can be formulated into tablets, capsules, powders, suspensions, solutions for oral administration and solutions for parenteral administration including intravenous, intradermal, intramuscular, and subcutaneous administration, and into solutions for application onto patches for transdermal application with common and conventional carriers, binders, diluents, and excipients.

While a chemical compound of the invention for use in therapy may be administered in the form of the raw chemical compound, it is preferred to introduce the active ingredient, optionally in the form of a physiologically acceptable salt, in a pharmaceutical composition together with one or more adjuvants, excipients, carriers, buffers, diluents, and/or other customary pharmaceutical auxiliaries.

In a preferred embodiment, the invention provides pharmaceutical compositions comprising the chemical compound of the invention, or a pharmaceutically acceptable salt or derivative thereof, together with one or more pharmaceutically acceptable carriers, and, optionally, other therapeutic and/or prophylactic ingredients, known and used in the art. The carrier(s) must be “acceptable” in the sense of being compatible with the other ingredients of the formulation and not harmful to the recipient thereof.

The invention further provides nutraceutical compositions comprising the chemical compound of the invention, or a pharmaceutically acceptable salt or derivative thereof, together with one or more nutraceutically acceptable carriers, and, optionally, other therapeutic and/or prophylactic ingredients, known and used in the art. The carrier(s) must be “acceptable” in the sense of being compatible with the other ingredients of the formulation and not harmful to the recipient thereof. An oral composition can generally include an inert diluent or an edible carrier. The nutraceutical composition can comprise a functional food component or a nutrient component. The term “functional food” refers to a food which contains one or a combination of components which affects functions in the body so as to have positive cellular or physiological effects. The term “nutrient” refers to any substance that furnishes nourishment to an animal.

Pharmaceutical compositions of the invention may be those suitable for oral, rectal, bronchial, nasal, pulmonal, topical (including buccal and sub-lingual), transdermal, vaginal or parenteral (including cutaneous, subcutaneous, intramuscular, intraperitoneal, intravenous, intraarterial, intracerebral, intraocular injection or infusion) administration, or those in a form suitable for administration by inhalation or insufflation, including powders and liquid aerosol administration, or by sustained release systems. Suitable examples of sustained release systems include semipermeable matrices of solid hydrophobic polymers containing the compound of the invention, which matrices may be in form of shaped articles, e.g. films or microcapsules.

The chemical compound of the invention, together with a conventional adjuvant, carrier, or diluent, may thus be placed into the form of pharmaceutical compositions and unit dosages thereof. Such forms include solids, and in particular tablets, filled capsules, powder and pellet forms, and liquids, in particular aqueous or non-aqueous solutions, suspensions, emulsions, elixirs, and capsules filled with the same, all for oral use, suppositories for rectal administration, and sterile injectable solutions for parenteral use. Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed.

The chemical compound of the present invention can be administered in a wide variety of oral and parenteral dosage forms. It will be obvious to those skilled in the art that the following dosage forms may comprise, as the active component, either a chemical compound of the invention or a pharmaceutically acceptable salt of a chemical compound of the invention.

For preparing pharmaceutical compositions from a chemical compound of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, solubilizers, lubricants, suspending agents, binders, preservatives, tablet disintegrating agents, or an encapsulating material.

In powders, the carrier is a finely divided solid, which is in a mixture with the finely divided active component. In tablets, the active component is mixed with the carrier having the necessary binding capacity in suitable proportions and compacted in the shape and size desired.

The powders and tablets preferably contain from five or ten to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

Liquid preparations include solutions, suspensions, and emulsions, for example, water or water-propylene glycol solutions. For example, parenteral injection liquid preparations can be formulated as solutions in aqueous polyethylene glycol solution. The chemical compound according to the present invention may thus be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulation agents such as suspending, stabilizing and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Aqueous solutions suitable for oral use can be prepared by dissolving the active component in water and adding suitable colorants, flavors, stabilizing and thickening agents, as desired. Aqueous suspensions suitable for oral use can be made by dispersing the finely divided active component in water with viscous material, such as natural or synthetic gums, resins, methylcellulose, sodium carboxymethylcellulose, or other well known suspending agents.

For topical administration to the epidermis the chemical compound of the invention may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilising agents, dispersing agents, suspending agents, thickening agents, or coloring agents.

Compositions suitable for topical administration in the mouth include lozenges comprising the active agent in a flavored base, usually sucrose and acacia or tragacanth; pastilles comprising the active ingredient in an inert base such as gelatin and glycerine or sucrose and acacia; and mouthwashes comprising the active ingredient in a suitable liquid carrier.

Solutions or suspensions are applied directly to the nasal cavity by conventional means, for example with a dropper, pipette or spray. The compositions may be provided in single or multi-dose form. In compositions intended for administration to the respiratory tract, including intranasal compositions, the compound will generally have a small particle size for example of the order of 5 microns or less. Such a particle size may be obtained by means known in the art, for example by micronization.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packaged tablets, capsules, and powders in vials or ampoules. Also, the unit dosage form can be a capsule, tablet, cachet, or lozenge itself, or it can be the appropriate number of any of these in packaged form.

Tablets, capsules and lozenges for oral administration and liquids for intravenous administration and continuous infusion are preferred compositions. Solutions or suspensions for application to the nasal cavity or to the respiratory tract are preferred compositions. Transdermal patches for topical administration to the epidermis are preferred.

Further details on techniques for formulation and administration may be found in the latest edition of Remington's Pharmaceutical Sciences (Maack Publishing Co., Easton, Pa.).

In another aspect the invention provides a method for the treatment, prevention or alleviation of obesity in a subject in need thereof, and which method comprises administering to such a subject, including a human, in need thereof an effective amount of the pterostilbene of the invention.

A therapeutically effective dose refers to that amount of active ingredient, which ameliorates the symptoms or condition. Therapeutic efficacy and toxicity, e.g. ED₅₀ and LD₅₀, may be determined by standard pharmacological procedures in cell cultures or experimental animals. The dose ratio between therapeutic and toxic effects is the therapeutic index and may be expressed by the ratio LD₅₀/ED₅₀. Pharmaceutical compositions exhibiting large therapeutic indexes are preferred.

The dosage of compound used in accordance with the invention varies depending on the compound and the condition being treated. The age, lean body weight, total weight, body surface area, and clinical condition of the recipient patient; and the experience and judgment of the clinician or practitioner administering the therapy are among the factors affecting the selected dosage. Other factors include the route of administration, the patient's medical history, the severity of the disease process, and the potency of the particular compound. The dose should be sufficient to ameliorate symptoms or signs of the disease treated without producing unacceptable toxicity to the patient. The dosage may be varied by titration of the dosage to the particular circumstances of this invention to produce the desired therapeutic effect.

Appropriate conversion of drug doses from animal studies to human studies (human equivalent dose, HED) is obtained through the use of the body surface area (BSA) normalization method. The interrelationship of dosages for animals and humans (based on milligrams per meter squared of body surface) is described by Reagan-Shaw et al. (2007. FASEB J. 22: 659-661). The formula for dose translation from animal dose to human dose through normalization to BSA (mg/m²) is:

$$HED ( mg \text{/} kg ) = Animal dose ( mg \text{/} kg ) multiplied by \frac{Animal {K}_{m}}{Human {K}_{m}}$$

where the Kₘ factor, body weight (kg) divided by BSA (m²), is used to convert the mg/kg dose used in the animal study to an mg/m². The Kₘ factor for rat is 6 and the Kₘ factor for human is 37. A table (Table 1, Reagan-Shaw et al., supra) lists Kₘ factors calculated for several animal species based on data from FDA Guidelines.

The pterostilbene is present in the composition in an amount sufficient to treat, alleviate, or prevent obesity in a subject in need thereof. In a most preferred embodiment, the pterostilbene is present in the composition in an amount sufficient to treat, alleviate, or prevent obesity by itself. The active ingredient may be administered in one or several doses per day. A satisfactory result can, in certain instances, be obtained at a dosage as low as the human equivalent dose (HED) of 1.6216 mg/kg p.o. or a dose of about 97 mg/day p.o. for a 60 kg human patient to a dose of about 291 mg/day p.o. for a 60 kg human patient. Given that pterostilbene has a half-life of approximately 2 hr, an appropriate range can be from about 10 mg/day p.o. to about 100 mg/day p.o. for said human patient.

It is at present contemplated that suitable dosage ranges are 10-100 mg daily for human patients, dependent as usual upon the exact mode of administration, form in which administered, the indication toward which the administration is directed, the subject involved and the body weight and body surface area of the subject involved, and further the preference and experience of the physician or veterinarian in charge. When administered in combination with compounds known in the art for treatment of the diseases, the dosing regimen may be reduced.

### EXAMPLES

Having now generally described this invention, the same will be better understood by reference to certain specific examples, which are included herein only to further illustrate the invention and are not intended to limit the scope of the invention as defined by the claims.

### Example 1

### Pterostilbene

Pterostilbene was synthesized as previously described (Joseph et al., supra). Briefly, pterostilbene was synthesized by condensation of 3,5-dimethoxybenzaldehyde and 4-hydroxyphenylacetic acid in acetic anhydride and triethylamine. The reaction mixture was heated (150° C.) under an atmosphere of nitrogen and continuously stirred. After 20 h, the reaction was stopped and cooled to room temperature, and concentrated hydrochloric acid (5 mL) was added. A precipitate formed, and this was dissolved in 50 mL of chloroform and then extracted with 10% aqueous sodium hydroxide. The aqueous extract was acidified to pH 1 with concentrated hydrochloric acid and stirred for at least 6 h, resulting in the precipitation of the intermediate product, α-[(3,5-dimethoxyphenyl)methylene]-4-hydroxy-(αZ)-benzeneacetic acid. This intermediate product was heated with 1.0 g of copper in 10 mL of quinoline (200° C., 6 h, under nitrogen). The reaction mixture was cooled to room temperature and filtered. To the filtrate was added 5 N hydrochloric acid (25 mL), which was stirred for 1 h and then extracted with chloroform. The chloroform extract containing impure pterostilbene was purified by flash chromatography on a Horizon HPFC system (Biotage, Inc., Charlottesville, Va.), using a silica gel column and the solvent system ethyl acetate:hexane (linear gradient from 15:85 to 100% ethyl acetate). Fractions containing pure pterostilbene were combined and concentrated in vacuum. Pterostilbene was recrystallized in hexane, and its structure was confirmed from its spectroscopic data (UV, mass spectrometry, and nuclear magnetic resonance spectroscopy) (FIG. 1).

### Example 2

### Effect of Pterostilbene on Body Weight, Food Intake, White Adipose Tissue Weights

The experiment was conducted with twenty seven male Wistar rats with an initial body weight of 180±2 grams purchased from Harlan Ibérica (Barcelona, Spain), and took place in accordance with the University of the Basque Country's guide for the care and use of laboratory animals (Reference protocol approval CUEID CEBA/30/2010). The rats were individually housed in polycarbonate metabolic cages (Techniplast Gazzada, Guguggiate, Italy) and placed in an air-conditioned room (22±2° C.) with a 12 h light-dark cycle. After a 6-day adaptation period, rats were randomly divided in 3 dietary groups of nine animals each, and fed on a commercial obesogenic diet, high in sucrose (20.0%) and fat (22.5%) (Harlan Iberica, TD.06415). Pterostilbene was added to the diet. Rats in the pterostilbene groups received 15 mg/kg body weight/d (PT15 group) or 30 mg/kg body weight/d (PT30 group). All animals had free access to food and water. Food intake and body weight were daily measured.

At the end of the 6-week experimental period, animals were sacrificed, under anaesthesia (chloral hydrate), by cardiac exsanguination. White adipose tissue from different anatomical locations (perirenal, epididymal, mesenteric and subcutaneous) and liver were dissected, weighed and immediately frozen.

No statistical differences in food intake and final body weight were found among experimental groups. Nevertheless, pterostilbene significantly reduced total adipose tissue mass (15.1% in PT15 group and 22.9% in PT30 group). With regard to specific anatomical locations, whereas in PT15 group only the subcutaneous depot was significantly affected, in PT30 groups perirenal, mesenteric and subcutaneous locations were reduced (Table I).

TABLE I
Effect of pterostilbene on adipose tissue and gastrocnemious muscle weights.

| | Control | PT15 | PT30 | ANOVA |
|---|---|---|---|---|
| Final Body Weight | 382 ± 5 | 382 ± 5 | 382 ± 5 | NS |
| Body Weight Increase (g) | 202 ± 10 | 206 ± 9 | 200 ± 6 | NS |
| Food Intake (g/d) | 17.6 ± 0.3 | 17.7 ± 0.5 | 17.5 ± 0.3 | NS |
| Adipose Tissue Weights (g) | | | | |
| Interscapular Brown | 0.74 ± 0.05 | 1.01 ± 0.23 | 0.75 ± 0.05 | NS |
| Epididymal | 11.8 ± 0.9 | 10.5 ± 0.9 | 10.5 ± 1.1 | NS |
| Perirenal | 13.5 ± 0.8^{a} | 12.0 ± 1.1^{ab} | 11.2 ± 1.0^{b} | P < 0.05 |
| Mesenteric | 6.0 ± 0.42^{a} | 5.2 ± 0.4^{ab} | 4.8 ± 0.5^{b} | 0.05 |
| Subcutaneous | 16.8 ± 1.1^{a} | 12.6 ± 1.2^{b} | 12.7 ± 1.6^{b} | P < 0.05 |
| Total Fat Depots | 47.5 ± 3.0^{a} | 40.3 ± 3.0^{b} | 36.6 ± 3.5^{b} | P < 0.05 |
| Gastrocnemious Muscles (2) (g) | 2.36 ± 0.08 | 2.48 ± 0.10 | 2.44 ± 0.08 | NS |

Values are means ± SEM (n = 9).
Values in the same row with different superscript are significantly different at P < 0.05 as determined by Newman-Keuls test.
NS, not significant.

### Example 3

### Effect of Pterostilbene on Enzyme Activities in Adipose Tissue and Liver

The activity of lipogenic enzymes in epididymal adipose tissue and liver, lipoprotein lipase in epididymal adipose tissue, and enzymes involved in fatty acid oxidation in the liver were assessed as previously described (Alberdi et al. 2011. Nutr. Metab. 8:29-35; Miranda et al. 2009. J. Am. Coll. Nutr. 28:43-49).

Both groups of rats fed the pterostilbene-supplemented diet showed significantly reduced activities of malic enzyme (ME) and fatty acid synthase (FAS) in epididymal adipose tissue. By contrast, the activity of lipoprotein lipase remained unchanged (Table II).

TABLE II
Effect of pterostilbene on adipose tissue lipogenic
enzymes and lipoprotein lipase.

| | Control | PT15 | PT30 | ANOVA |
|---|---|---|---|---|
| G6PDH | 56.65 ± 7.72 | 62.96 ± 8.54 | 44.78 ± 2.51 | NS |
| activity¹ | | | | |
| ME | 84.08 ± 10.11^{a} | 51.00 + 5.47^{b} | 42.54 ± 5.47^{b} | P < 0.01 |
| activity¹ | | | | |
| FAS | 35.66 ± 3.60^{a} | 17.95 ± 3.42^{b} | 17.34 ± 4.12^{b} | P < 0.01 |
| activity² | | | | |
| HR-LPL | 1.75 ± 0.20 | 1.64 ± 0.30 | 1.75 ± 0.26 | NS |
| activity³ | | | | |

Values are means ± SEM (n = 9).
Values in the same row with different superscript are significantly different at P < 0.05 as determined by Newman-Keuls test.
G6PDH: glucose-6-phosphate dehydrogenase;
ME: malic enzyme;
FAS: fatty acid synthase;
HR-LPL: heparin releasable lipoprotein lipase;
NS, not significant
¹nmol NADPH formed/mg protein/min
²nmol NADPH consumed/mg protein/min
³nmol oleate released per minute per gram of tissue

Similar to that observed in epididymal adipose tissue, the activity of ME in the liver was reduced in both groups (Table III). In the PT30 group, activity of glucose-6-phosphate dehydrogenase (G6PDH) was also significantly reduced. While not statistically significant, the activity of FAS was decreased (see FIG. 2). The activities of liver enzymes involved in fatty acid oxidation (carnitine palmitoyltransferase and acyl-coenzyme A oxidase) were significantly increased in the PT30 group (Table IV).

TABLE III
Effect of pterostilbene on hepatic lipogenic enzyme activity.

| | Control | PT15 | PT30 | ANOVA |
|---|---|---|---|---|
| G6PDH | 28.00 ± 4.17^{a} | 19.66 ± 1.75^{ab} | 15.87 ± 0.99^{b} | P < 0.05 |
| activity¹ | | | | |
| ME | 22.65 ± 2.33^{a} | 16.80 ± 1.54^{b} | 16.00 ± 1.49^{b} | P < 0.05 |
| activity¹ | | | | |
| FAS | 5.42 ± 1.04 | 3.71 ± 0.57 | 3.33 ± 0.37 | NS |
| activity² | | | | |

Values are means ± SEM.
Values in the same row with different superscript are significantly different at P < 0.05 as determined by Newman-Keuls test.
G6PDH: glucose-6-phosphate dehydrogenase;
ME: malic enzyme;
FAS: fatty acid synthase;
NS, not significant
¹nmol NADPH formed/mg protein/min
²nmol NADPH consumed/mg protein/min

TABLE IV
Effect of pterostilbene on hepatic carnitine palmitoyl
transferase and acyl-CoA oxidase.

| | Control | PT15 | PT30 | ANOVA |
|---|---|---|---|---|
| CPT-Ia | 6.48 ± 1.10^{a} | 8.28 ± 1.08^{ab} | 10.36 ± 0.39^{b} | P < 0.05 |
| activity¹ | | | | |
| ACO | 4.17 ± 0.69^{a} | 4.54 ± 0.12^{a} | 7.34 ± 0.50^{b} | P < 0.01 |
| activity² | | | | |

Values are means ± SEM.
Values in the same row with different superscript are significantly different at P < 0.05 as determined by Newman-Keuls test.
CPT: carnitine pallmitoyltransferase;
ACO: acyl-CoA oxidase
¹nmol CoA formed/min/mg protein
²nmol NADH formed/min/mg protein

### Example 4

### Serum and Tissue Levels of Pterostilbene

Extraction of pterostilbene in the serum was according to published procedure (Kotani et al. 2003. J. Chromatogr. B. 788:269-275), with modifications. Serum from −80° C. storage was thawed on ice. To 50 μL serum was added 15 μL of β-glucuronidase (from E. coli, Type IX-A, Sigma-Aldrich, St. Louis, Mo., USA; 250 U/15 mL sodium phosphate buffer, 0.1M, pH 6.8) and 15 μL of sulfatase (from Abalone Entrails, Type VIII, Sigma-Aldrich, St. Louis, Mo., USA; 20 U/15 mL sodium phosphate buffer, 0.1M, pH 6.8), then vortex-mixed. This mixture was incubated for 20 hr at 37° C. while shaking at 750 rpm, cooled to room temperature, then partitioned with ethyl acetate (75 μL×3). The ethyl acetate layers were combined and dried under nitrogen. The dried sample was derivatized with 40 μL of N,O-bis[trimethylsilyl]-trifluoroacetamide: dimethyl-formamide (BSTFA:DMF, 1:1; Pierce Biotechnology, Inc., Rockford, Ill.), heated at 70° C. for 40 min and used for the analysis of pterostilbene by gas chromatography-mass spectrometry (GC-MS).

Pterostilbene in liver and muscle was extracted following published procedure (Hou et al. 2011. Planta Med. 77:455-460), with minor modifications. For liver, 100 mg fresh tissues were homogenized in 1 mL saline solution (0.9% NaCl in H₂O). For muscle, 100 mg lyophilized tissue was homogenized in 1 mL sodium acetate buffer, pH 5. To the homogenates was added 300 μL enzyme solution (793.67 U/mL sulfatase and 666.66 U/mL β-glucuronidase in sodium acetate buffer, pH 5), vortex-mixed, and incubated at 37° C. for 16 hr while shaking at 750 rpm. To this mixture was added 100 μL of 0.1N HCl, then partitioned with ethyl acetate (800 μL×2). The ethyl acetate layers were combined, then dried under nitrogen. The dried sample was derivatized with BSTFA:DMF under the same conditions used for the analysis of pterostilbene in serum.

For the analysis of pterostilbene in epididymis and subcutaneous tissues, 100 mg of lyophilized tissue was homogenized in 300 μL of sodium acetate buffer, then sonicated for 30 min. To the homogenate was added 300 μl enzyme solution (793.67 U/mL sulfatase and 1333.33 U/mL β-glucuronidase in sodium acetate buffer, pH5) vortex-mixed, and incubated at 37° C., with shaking (750 rpm), for 16 hours. After incubation, 500 μl ice-cold acetonitrile was added, vortex-mixed for 1 min, then centrifuged at 800 rpm (Savant SpeedVac, model number SPD121P-120) for 20 min. The clear supernatant was collected, and again treated with 500 μl ice-cold acetonitrile, vortex-mixed and then centrifuged at 800 rpm for 20 min. The supernatant was then dried under a stream of nitrogen, then derivatized with BSTFA:DMF using the same conditions as in the analysis of pterostilbene in serum.

GC-MS analysis of pterostilbene in serum and tissues was performed using a J&W DB-5 capillary column (0.25 mm internal diameter, 0.25 μm film thickness, 30 m length; Agilent Technologies, Foster City, Calif.) on a JEOL GCMate II Instrument (JEOL USA Inc., Peabody, Mass.). For the serum samples, the GC temperature program was: initial 180° C. held at this temp for 1 min, increased to 264° C. at 12° C./min rate and held at this temp for 4 min, increased to 300° C. at the rate of 30° C./min and held at this temperature for 0.5 min. The retention time of pterostilbene was 9.7 min. For the tissue samples, the GC temperature program was: initial 180° C. held at this temp for 1 min, increased to 263° C. at 14° C./min rate and held at this temp for 5 min, increased to 300° C. at the rate of 35° C./min and held at this temperature for 0.5 min. The retention time of pterostilbene was 8.6 min. The carrier gas was ultrahigh purity helium, at 1 mL/min flow rate. The injection port, GC-MS interface, and ionization chamber were at 250, 230, and 230° C., respectively. The volume of injection was 1 μL, splitless injection. The mass spectrum was acquired in positive, electron impact (70 eV), low-resolution, selected ion monitoring mode using masses m/z 328, 313, and 296. Quantitation was performed from a calibration curve of a standard sample of pterostilbene.

Results are presented as means±standard error of the means. Statistical analysis was performed using SPSS 17.0 (SPSS Inc. Chicago, Ill., USA). Data were analyzed by Student's t test. Statistical significance was set-up at the P<0.05 level.

Pterostilbene was detected in serum, liver, epididymis, and subcutaneous tissues in both the PT15 and PT30 animals, with the PT15 showing lower levels than the PT30 group. In muscles, pterostilbene was found only in the PT30 group (Table V).

TABLE V
Serum and tissue Levels of pterostilbene.

| | PT15 | | PT30 | |
|---|---|---|---|---|
| Serum | 384.03 ± 54.68 | ng/mL^{a} | 788.68 ± 246.46 | ng/mL^{b} |
| Liver | 30.80 ± 11.70 | ng/100 mg FW^{a} | 72.58 ± 11.19 | ng/100 mg FW^{b} |
| Epididymis | 1.09 ± 0.53 | ng/100 mg DW^{a} | 3.07 ± 1.10 | ng/100 mg DW^{a} |
| Subcutaneous | 2.05 ± 1.37 | ng/100 mg DW^{c} | 3.54 ± 0.77 | ng/100 mg DW^{c} |

| | | | |
|---|---|---|---|
| Muscle | Not Detected | 0.91 ± 0.20 | ng/100 mg DW^{a} |

^{a}n = 8;
^{b}n = 6;
^{c}n = 7
FW: fresh weight;
DW: dry weight

All publications and patents mentioned in this specification are herein incorporated by reference to the same extent as if each individual publication or patent was specifically and individually indicated to be incorporated by reference.

It is understood that the foregoing detailed description is given merely by way of illustration and that modifications and variations may be made therein without departing from the spirit and scope of the invention.

1. A method for treating or alleviating obesity in an individual comprising administering to an individual in need thereof a therapeutically effective amount of a pharmaceutical or nutraceutical composition consisting essentially of isolated or chemically-synthesized pterostilbene, its pharmaceutically or nutraceutically acceptable salts or isomers thereof and a pharmaceutically or nutraceutically acceptable carrier, wherein the composition is effective to treat obesity in the individual.

2. A method of decreasing the total adipose tissue mass in an individual comprising administering to an individual in need thereof a therapeutically effective amount of a pharmaceutical or nutraceutical composition consisting essentially of isolated or chemically-synthesized pterostilbene, its pharmaceutically or nutraceutically acceptable salts or isomers thereof and a pharmaceutically or nutraceutically acceptable carrier, wherein the composition is effective to decrease the total adipose tissue mass in the individual.

3. A method of reducing adipose depots in anatomical locations in an individual comprising administering to an individual in need thereof a therapeutically effective amount of a pharmaceutical or nutraceutical composition consisting essentially of isolated or chemically-synthesized pterostilbene, its pharmaceutically or nutraceutically acceptable salts or isomers thereof and a pharmaceutically or nutraceutically acceptable carrier, wherein the composition is effective to reduce adipose depots in anatomical locations in the individual.

4. The method of claim 3 wherein said anatomical locations of adipose depots are depots in subcutaneous and/or mesenteric and/or epididymal and/or perirenal locations.

5. A method of inhibiting lipogenesis in an individual comprising administering to an individual in need thereof a therapeutically effective amount of a pharmaceutical or nutraceutical composition consisting essentially of isolated or chemically-synthesized pterostilbene, its pharmaceutically or nutraceutically acceptable salts or isomers thereof and a pharmaceutically or nutraceutically acceptable carrier, wherein the composition is effective to inhibit lipogenesis in the individual.

6. A method of increasing fatty acid oxidation in the liver of an individual comprising administering to an individual in need thereof a therapeutically effective amount of a pharmaceutical or nutraceutical composition consisting essentially of isolated or chemically-synthesized pterostilbene, its pharmaceutically or nutraceutically acceptable salts or isomers thereof and a pharmaceutically or nutraceutically acceptable carrier, wherein the composition is effective to increase fatty acid oxidation in the liver of the individual.

7. The method of any one of claims 1-3, 5 and 6 wherein said amount is a total amount and is based on the body weight and the body surface area of said subject.

8. The method of any one of claims 1-3, 5 and 6 wherein said amount is a total amount of about 100 mg/day to about 500 mg/day.

9. The method of claim 1 wherein said nutraceutical composition is a food or beverage or a supplement composition for a food or beverage.

10. A kit for treating or alleviating obesity, wherein the kit comprises:
a composition consisting essentially of an effective amount of pterostilbene according to claim 1, a container housing the composition; and instructions for the use of the kit.
